(19) United States
(12) Patent Application Publication
(10) Pub. No.: US 2023/0273164 A1
(43) Pub. Date: Aug. 31, 2023

(54) COMPOSITIONS, APPARATUS, AND METHODS FOR DETERMINING PHOSPHATE CONTENT OF WATER

(71) Applicant: WATER LENS, LLC, Houston, TX (US)

(72) Inventors: Adam GARLAND, Houston, TX (US); Tiffany KIM, Houston, TX (US); Cody HONEY, Houston, TX (US); Tyler WEST, Houston, TX (US)

(21) Appl. No.: 18/001,600

(22) PCT Filed: Jun. 15, 2021
(86) PCT No.: PCT/US2021/037418

§ 371 Date: Dec. 13, 2022

Related U.S. Application Data
(60) Provisional application No. 63/039,254, filed on Jun. 15, 2020.

(51) Int. Cl. G01N 31/22; G01N 21/78; G01N 33/18; B01L 3/00

(52) U.S. Cl. CPC G01N 31/22; G01N 21/78; G01N 33/18; B01L 3/50855; B01L 2200/16

(57) ABSTRACT

Compositions, kits and methods of using the kits and compositions to determine the phosphate concentration in a solution is described. The composition comprising: an indicator, a molybdate salt, a buffer, a reaction accelerant, a sulfate salt, and one or more excipient, wherein the composition has an absorbance at a detectable wavelength in response to the phosphate concentration in a solution. The kit can include a lyophilized composition which has absorbance at a detectable wavelength in response to the phosphate concentration in the solution.

### CROSS REFERENCE TO RELATED APPLICATIONS

This application claims the benefit of U.S. Provisional Application No. 63/039,254, filed Jun. 15, 2020. The contents of the referenced application are incorporated into the present application by reference.

### BACKGROUND OF THE INVENTION

### A. Field of the Invention

The invention generally concerns determining the phosphate concentration in a target/analyte composition. In particular, the kits and methods of the present invention can be used to determine the phosphate concentration in an analyte composition by adding the analyte composition to a plurality of lyophilized compositions, which can produce a plurality of solutions. The plurality of solutions can then be used to calculate the phosphate concentration in the analyte composition.

### B. Description of Related Art

Determining the phosphate concentration of various aqueous liquids is important for several manufacturing and environmental purposes. For example, the concentration of phosphate in agricultural runoff must be carefully regulated to prevent unwanted blooms in algal and bacterial species. Phosphate concentration is also monitored in municipal and industrial waters to prevent or reduce corrosion in piping networks. Current methods to test phosphate concentrations at remote sites include test strips or wet chemistry kits. EPA methods 365.1, 365.2, and 365.3 for determining phosphate concentration by wet chemistry utilize a derivative of the phosphomolybdate method. One widely used method for phosphate determination is the malachite green method, which is a derivative of the phosphomolybdate method. While potentially accurate with careful execution, this method suffers from several disadvantages and is time consuming. First, it requires the use of high concentration acid (typically >3M sulfuric acid) to improve the solubility of the malachite green. This presents several shipping, handling, and safety issues which must be taken into consideration during implementation. It also makes formulating quick dissolving solids, such as lyophilized powders, exceedingly difficult. Second, the method is sensitive to interferences from several common anions including sulfate and nitrate, some of which cause significant change to the output of the assay. Third, the assay takes at least 3 minutes to fully develop. Taking measurements before the proper time has elapsed can result in false negatives.

### SUMMARY OF THE INVENTION

A solution to at least some of the disadvantages described above and associated with the standard malachite green method has been discovered. In one aspect, a solution of the present invention provides for the use of a colorimetric assay that includes a plurality lyophilized composition samples positioned in individual microwells of a microwell plate. The plurality of lyophilized compositions can include a colorimetric indicator that has an absorbance at a detectable wavelength in response to the concentration of phosphate in a solution that is to be analyzed (e.g., the analyte solution/composition/sample). Analyte samples are added to the lyophilized samples and the absorbance of the resulting samples is measured and the phosphate concentration of the analyte composition can be determined based on the measured absorbance value. Notably, an advantage of the present invention is that it can eliminate or reduce the drawbacks of the aforementioned standard malachite green assay. For example, the kits and methods of the present invention can (1) reduce or eliminate the need for using high concentration acid (typically >3M sulfuric acid), (2) reduce or eliminate interferences from several common anions including sulfate and nitrate that may also be present in the analyte composition, and/or (3) can be performed in a quicker manner when compared with the typical time of at least 3 minutes for the standard malachite green assay. With respect to (3), the methods and kits of the present invention can accurately identify the phosphate concentration of an analyte composition in less than 180 seconds, less than 120 seconds, or less than 60 seconds, preferably about 30 seconds.

In one aspect of the invention, there is disclosed a composition for determining the phosphate concentration in an analyte solution. The composition can include an indicator, a molybdate salt, a buffer, a reaction accelerant, a sulfate salt, and one or more excipients, wherein the composition can have an absorbance at a detectable wavelength in response to the phosphate concentration in the solution. The indicator can include one or more of methyl violet 2B, methyl violet 6B, methyl violet 10B, pararosaniline, fuchsine, new fuchsine, fuchsine acid, phenolphthalein, phenol red, chlorophenol red, cresol red, bromocresol purple, bromocresol green, bromophenol blue, pyrogallol red, malachite green, brilliant green, brilliant blue FCF, Victoria blue B, Victoria blue FBR, Victoria blue BO, Victoria blue FGA, Victoria blue 4 R, Victoria blue R, eosin B, eosin Y, rhodamine B, rhodamine 123, and fluorescein, with malachite green being preferred. The molybdate salt includes one of more of ammonium molybdate, ammonium molybdate tetrahydrate, sodium molybdate, sodium molybdate dihydrate, ceric ammonium molybdate, cerium molybdate, potassium molybdate, magnesium molybdate, lithium molybdate, calcium molybdate, zinc molybdate, bismuth molybdate, lead molybdate, molybdic acid, nickel molybdate, silver molybdate, strontium molybdate, barium molybdate, and cadmium molybdate, with ammonium molybdate being preferred. The reaction accelerant includes one or more of sodium nitrate, lithium nitrate, nitric acid, potassium nitrate, magnesium nitrate, calcium nitrate, strontium nitrate, barium nitrate, and ammonium nitrate, with sodium nitrate being preferred. The sulfate salt includes one or more of sodium sulfate, potassium sulfate, lithium sulfate, sulfuric acid, magnesium sulfate, and calcium sulfate, with sodium sulfate being preferred. The buffer includes one or more of camphorsulfonate, camphorsulfonic acid, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, selenocysteine, pyrrolysine, and valine, with valine being preferred. A reducing agent can also be included in the composition. The reducing agent can include one or more metal or metal containing compounds (e.g., potassium, calcium, barium, sodium, or magnesium or compounds having such metals) or compounds that contain the H— ion (e.g., NaH, LiH, LiAlH₄ and CaH₂). A redox catalyst can also be included in the composition. The redox catalyst can include one or more of transition metals or compounds comprising transition metals (e.g., Groups 3 through 12 metals). Some non-limiting examples include iron, ferrous iron salts, ferric iron salts, Fe(II)-EDTA complex, Fe(III)-EDTA complex, Fe(II)-CDTA complex, or Fe(III)-CDTA complex). The excipients include one or more of a polyethylene glycol, a 1,1,3,3-tetramethylbutyl) phenyl-polyethylene glycol, (2-hydroxylpropyl)-β-cyclodextrin, glycine, cellulose, citrate, lactose, mannitol, xylitol, sucrose, and polyvinylpyrrolidone, with (2-hydroxylpropyl)-β-cyclodextrin being preferred. The composition can be a powder. The powder can be made by providing an aqueous solution of the composition to one or more containers and subjecting at least one of the containers to lyophilizing conditions sufficient to remove the water from the aqueous solution to form the powder. In some instances, the one or more containers are microwells of a microwell plate. The powder can be packaged (for example, a bag, vial, or encapsulated).

In one aspect of the invention, there is disclosed a phosphate assay kit. The kit can include a) a microwell plate and b) a lyophilized composition including an indicator, a molybdate salt, a buffer, a reaction accelerant, a sulfate salt, and one or more excipients. A plurality of microwells (e.g., 2, 3, 4, 5, 6, 7, 8, 9, 10, 11, 12, 13, 14, 15, 16, 17, 18, 19, 20, 24, 30, 35, 40, 45, 48, 50, 55, 60, 65, 70, 75, 80, 85, 90, 96, or more microwells) of the microwell plate can contain the lyophilized composition such that when an analyte composition is added to the lyophilized composition in each microwell of the plurality of microwells a solution forms in each individual microwell, wherein each solution can have a absorbance at detectable wavelengths in response to phosphate comprised in the analyte composition. By developing calibration curves based on these proportions, the phosphate concentration can be quickly determined quantitatively. The detectable wavelengths can be 400 nm, 425 nm, 450 nm, 475 nm, 500 nm, 525 nm, 550 nm, 575 nm, 600 nm, 625 nm, 650 nm, 675 nm, or 700 nm or any range therein. In some aspects a preferred wavelengths are 440-460 nm, 510-530 nm, 550-560 nm, 580-600 nm, and 610-630 nm, or more preferably 450 nm, 520 nm, 560 nm, 595 nm, and 620 nm. In some preferred aspects, the microwell plate can include 6, 24, 96, 384, or 1536 microwells. In some aspects of the invention, the microwell plate includes at least 8 microwells and each microwell contains the same composition or at least 2 microwells have the same composition and the rest of the microwells have different amounts of the same components In other aspects of the invention, the microwell plate has at least 24 or 96 microwells and some of the microwells have different amounts of the same composition or of the same components when compared with the other microwells. In some aspects, a plurality of the microwells can have the same composition but different amounts of the same composition. In some aspects, a plurality of the microwells can have the same components but different amounts of the same components. In some aspects, there can be at least a first set of a plurality of microwells and at least a second set of a plurality of microwells, wherein the first and second sets can each have the same composition or same components, but the first set can have an increased amount of the compositions or components when compared with the second set or the second set can have an increased amount of the compositions or components when compared with the first set. The indicator can include one or more of methyl violet 2B, methyl violet 6B, methyl violet 10B, pararosaniline, fuchsine, new fuchsine, fuchsine acid, phenolphthalein, phenol red, chlorophenol red, cresol red, bromocresol purple, bromocresol green, bromophenol blue, pyrogallol red, malachite green, brilliant green, brilliant blue FCF, Victoria blue B, Victoria blue FBR, Victoria blue BO, Victoria blue FGA, Victoria blue 4 R, Victoria blue R, eosin B, eosin Y, rhodamine B, rhodamine 123, and fluorescein, with malachite green being preferred. The molybdate salt can include one of more of ammonium molybdate, ammonium molybdate tetrahydrate, sodium molybdate, sodium molybdate dihydrate, ceric ammonium molybdate, cerium molybdate, potassium molybdate, magnesium molybdate, lithium molybdate, calcium molybdate, zinc molybdate, bismuth molybdate, lead molybdate, molybdic acid, nickel molybdate, silver molybdate, strontium molybdate, barium molybdate, and cadmium molybdate, with ammonium molybdate being preferred. The reaction accelerant can include one or more of sodium nitrate, lithium nitrate, nitric acid, potassium nitrate, magnesium nitrate, calcium nitrate, strontium nitrate, barium nitrate, and ammonium nitrate, with potassium nitrate being preferred. The sulfate salt includes one or more of sodium sulfate, potassium sulfate, lithium sulfate, sulfuric acid, magnesium sulfate, and calcium sulfate, with sodium sulfate being preferred. The buffer can include one or more of camphorsulfonate, camphorsulfonic acid, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, selenocysteine, pyrrolysine, and valine, with valine being preferred. The excipient(s) can include one or more of a polyethylene glycol, a 1,1,3,3-tetramethylbutyl) phenyl-polyethylene glycol, (2-hydroxylpropyl)-β-cyclodextrin, glycine, cellulose, citrate, lactose, mannitol, xylitol, sucrose, and polyvinylpyrrolidone, with (2-hydroxylpropyl)-β-cyclodextrin being preferred. In a preferred aspect, the lyophilized composition includes ammonium molybdate, malachite green, potassium nitrate, sodium sulfate, (2-hydroxylpropyl)-β-cyclodextrin, and valine. The plurality of microwells can be sealed to prevent the composition from exiting the plurality of microwells. In some instances, the plurality of microwells is sealed with a plastic film or a foil. The phosphate assay kit can also include a spectrophotometer capable of measuring ultra violet and visible wavelengths.

Also disclosed are methods to use the phosphate assay kit of the present invention to determine the phosphate concentration of an analyte composition or a plurality of analyte compositions. The method can include a) obtaining any one of the phosphate assay kits described throughout this Specification; b) obtaining an analyte composition; c) adding substantially the same or the same volume of the analyte composition to each of the plurality of microwells of the microwell plate to form solutions from the analyte composition and the lyophilized titrate compositions in each of the plurality of microwells; and d) measuring the absorbance value for each solution in each of the plurality of microwells at a wavelength and determining the phosphate of the analyte composition based on the measured absorbance values in response to the phosphate of the analyte composition. The analyte can be an aqueous sample from a variety of sources such as a subsurface water well in a hydrocarbon formation, a wastewater storage unit, a boiler, cooling unit, a wastewater reservoir, a tank, a water well, a lake, a river, an ocean, a canal, a pond, a pool, rainwater, agricultural runoff, ballast water, bilge water, mining runoff, food processing waste water, blowdown water, brackish water, municipal drinking water, or the like. The analyte can be an aqueous solution or suspension such as blood, plasma, lymph, urine, saliva, milk, juice, beer, wine, spirits, liqueur, soft drink, sports drink, soup, stew, broth, bottled water, tap water, tea, coffee, electrolyte solution, saline solution, hydraulic fracturing fluid, chemical formulation, coolant, cleaning solution, aqueous extract, fertilizer solution, culture media, or the like. In some instances, the analyte composition is obtained from a hydrocarbon drilling or fracking process. In some instances, a plurality of solutions having the same analyte is obtained, and each analyte composition is obtained from a different source, well, plurality of subsurface wells, or a plurality of different water sources. In some instances, the analyte composition is obtained from a hydrocarbon drilling or fracking process. In some instances, a plurality of analyte compositions having the same analyte (e.g., phosphorous or phosphorous containing salts or compounds) can be obtained, and each analyte composition can be obtained from the same or from a different well of a plurality of subsurface wells or a plurality of different wastewater units.

The phosphate assay kits described throughout the Specification can be made by a) obtaining a microwell plate, b) obtaining a lyophilized composition including an indicator, a molybdate salt, a buffer, a reaction accelerant, a sulfate salt, and one or more excipients; wherein a plurality of microwells of the microwell plate contain the lyophilized composition such that when an analyte composition (e.g., a liquid analyte composition such as an aqueous analyte composition) is added to the lyophilized composition in each microwell of the plurality of microwells a solution can form in each microwell, wherein each solution can have an absorbance at a known wavelength in response to phosphate of the analyte composition. In some instances, the lyophilized composition can be obtained by providing an aqueous solution of the composition (the composition having an indicator, a molybdate salt, a buffer, a reaction accelerant, a sulfate salt, and one or more excipients) to one or more microwells of the microwell plate and subjecting the microwell plate to lyophilizing conditions sufficient to remove the water from the aqueous solution and form a powder. The plurality of microwells can be sealed with a plastic film or a foil to prevent the composition from exiting the plurality of microwells.

The term “acidic solution” or “acid compound” refers to a solution that has a concentration of hydrogen ions greater than the concentration of hydroxide ion ([H+]>[OH⁻]).

The terms “basic solution” or “alkaline solution” refers to a solution that has a concentration of hydrogen ions less than the concentration of hydroxide ion ([H+]<[OH⁻]).

The term “pH” refers to the measurement of the concentration of hydrogen ions in water or other media. pH is generally expressed as a log scale based on 10 where pH=−log[H+].

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The phosphate assay kits and the methods of using and making the phosphate assay kits of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the kits of the present invention is the ability to determine the phosphate concentration in an aqueous solution using spectrometric analysis.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIGS. 1A-1C are schematics of phosphate assay kits of the present invention.

FIG. 2 is a flow chart depicting a method of determining the phosphate concentration of a water body.

FIG. 3 is a graph of phosphate (in mg/L) versus absorbance.

### DETAILED DESCRIPTION OF THE INVENTION

Conventional implementations of the malachite green method require the use of high concentration acid (typically >3M sulfuric acid) to improve the solubility of the malachite green. This presents several shipping, handling, and safety issues which must be taken into consideration during implementation. It also makes formulating quick dissolving solids, such as lyophilized powders, exceedingly difficult. A discovery has been made in the context of the present invention, which allows for much less acid to be used in the assay formulation when compared with the traditional malachite green method. In one aspect, it was discovered that the use of (2-hydroxylpropyl)-β-cyclodextrin as an excipient in the lyophilized compositions of the present invention results in unexpected effects. For example, the normal color change of the assay becomes reversed, suggesting that the (2-hydroxylpropyl)-β-cyclodextrin is chemically involved in the color development process. Its use also can aid in the solubility of malachite green, permitting much higher pH values to be used for appropriate color development. While the standard application of the malachite green method runs at pH≤0, use of (2-hydroxylpropyl)-β-cyclodextrin allows the assay to function at pH≥1.5, with a preferred pH range of 1.0-2.5.

Additionally, the conventional malachite green method is sensitive to interferences from several common anions, some of which cause significant change to the output of the assay. A discovery has been made in the context of the present invention, which allows for the effects of many of these ions to be negated. The presence of small concentrations of sulfate≤1 μg/L cause a dramatic change to the response of the assay to the presence of phosphate. The effect appears not to be concentration dependent at higher concentrations, as it was essentially unchanged at sulfate concentrations≥1,000 mg/L. This small amount of sulfate added into the test solution during manufacturing makes the assay extremely resistant to interference caused by additional sulfate up to 10,000 mg/L of sulfate.

Further, the conventional malachite green assay takes at least 3 minutes to fully develop. Taking measurements before the proper time has elapsed results in false negatives. Formulations which use strong acid have faster kinetics, but at higher pH values the assay develops more slowly. Attempted formulations using pH≥1 took 15 minutes or longer after loading the sample to fully develop, with development times increasing as the phosphate concentration in the sample increases. A discovery has been made in the context of the present invention that accelerates the kinetics of the standard assay without the addition of strong acid. By way of example, the presence of nitrate appears to decrease the development time of the assay significantly. In one aspect, formulations including nitrate can reach full development within less than 3 minutes, within less than 2 minutes, within less than 1 minute, within 30 seconds, or within 15 second to 60 seconds, or within 15 seconds to 3 minutes, or within 15 seconds to 2 minutes without any negative side effects. In some aspects the formulations including nitrate were demonstrated to reach full development within 30 seconds without any negative side effects. Excessive concentrations of nitrate were found to be undesirable as they negatively affect freeze drying performance and shelf life.

Still further, the conventional malachite green assay runs in strong acid (usually sulfuric acid at concentration≥3M). To replace this strong acid while maintaining reaction consistency and production consistency, a buffer is typically employed. There are limited buffers which have pKₐ values near the target pH of 1-2.5, but amino acids fit the role well. Solubility issues were encountered when using most amino acids to hold the appropriate pH. It was discovered in the context of the present invention that amino acids with aliphatic side chains allowed significantly increased solubility of the solution components compared with other amino acids. By way of example, Valine was determined to provide the maximum solubility of reagents as well as optimal signal stability, assay kinetics, and signal to noise ratio in the final product.

Phosphate assays can be used to detect other phosphate containing species such as phosphate esters and phosphonates. Commonly these phosphate containing species are converted to ortho phosphate by chemical pretreatment. The chemicals used in these pretreatments, such as acids and oxidizers, can cause issues with the performance of the phosphate assay and can be neutralized prior to addition of the sample to the assay. Unfortunately, the types of chemicals used to neutralize the pretreatment chemicals also can cause issues with the performance of the phosphate assay. In addition, these neutralizing chemicals may require a catalyst to allow them to react fast enough. It was determined that a mixture of ascorbic acid as a neutralizer with Fe(III)-EDTA complex as a catalyst was capable of rapidly neutralizing pretreatment chemicals without compromising the performance of the phosphate assay.

In some aspects of the present invention, the reagents used to obtain optimal performance of the phosphate assay sometimes may not be as compatible with each other in solution. It was discovered that potentially incompatible reagents could be separated from each other during manufacturing by separately layering the reagent mixtures on top of each other prior to lyophilization without the layers mixing with each other. This technique can be used to reduce or prevent any incompatible reagents from reacting with each other prior to the assay being run without compromising the performance of the assay.

These and other non-limiting aspects of the present invention are discussed in further detail in the following sections.

### A. Composition

The composition can be made by preparing an aqueous solution of reagent solution and then subjecting the solution to lyophilizing conditions to remove the water and produce a powder. An aqueous solution of an indicator, a molybdate salt, a buffer, a reaction accelerant, a sulfate salt, and one or more excipients can be prepared. The indicator can be one or more of methyl violet 2B, methyl violet 6B, methyl violet 10B, pararosaniline, fuchsine, new fuchsine, fuchsine acid, phenolphthalein, phenol red, chlorophenol red, cresol red, bromocresol purple, bromocresol green, bromophenol blue, pyrogallol red, malachite green, brilliant green, brilliant blue FCF, Victoria blue B, Victoria blue FBR, Victoria blue BO, Victoria blue FGA, Victoria blue 4 R, Victoria blue R, eosin B, eosin Y, rhodamine B, rhodamine 123, and fluorescein. the molybdate salt comprises one or more of ammonium molybdate, ammonium molybdate tetrahydrate, sodium molybdate, sodium molybdate dihydrate, ceric ammonium molybdate, cerium molybdate, potassium molybdate, magnesium molybdate, lithium molybdate, calcium molybdate, zinc molybdate, bismuth molybdate, lead molybdate, molybdic acid, nickel molybdate, silver molybdate, strontium molybdate, barium molybdate, and cadmium molybdate. The buffer comprises one or more of camphorsulfonate, camphorsulfonic acid, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, selenocysteine, pyrrolysine, and valine. Suitable excipients of the composition include, but are not limited to, binders, diluents, disintegrants, detergents, surfactants, lubricants, glidants, carriers, and the like. A variety of materials may be used as fillers or diluents. The term “binder” in certain aspects refers to a substance that improves compression and promotes association between individual particles after compression. Binders can be used, for example, for dry granulation and direct compression, or dissolved in water or a solvent for use in wet granulation. Common binders include saccharides, gelatins, pregelatinized starches, microcrystalline cellulose, hydroxypropylcellulose and cellulose ethers, as well as polyvinylpyrrolidone (PVP). Suitable diluents or fillers include, but are not limited to, sucrose, dextrose, sorbitol, starch, cellulose (e.g. microcrystalline cellulose; Avicel®), dihydrated or anhydrous dibasic calcium phosphate, calcium carbonate, calcium sulfate, and others as known in the art. Suitable surfactants or detergents include nonionic surfactants, for example polyoxyethylene glycol alkyl ethers such as octaethylene glycol monododecyl ether and pentaethylene glycol monododecyl ether, polyoxypropylene glycol alkyl ethers, glucoside alkyl ethers, such as decyl glucoside, lauryl glucoside, and octyl glucoside, polyoxyethylene glycol octylphenyl ethers, such as Triton™ X-45, X-114, X-100, and X-102, polyoxyethylene glycol alkylphenyl ethers, such as nonoxynol-4, -9, -14, -15, 18, -30, and -50, glycerol alkyl esters, such as glyceryl laurate, polyoxyethylene glycol sorbitan alkyl esters, sorbitan alkyl esters, cocamide MEA, cocamide DEA, dodecyldimethylamine oxide, and block copolymers of polyethylene glycol, such as polyethylene-glycol (PEG) 300, 400, 1000, 1540, 4000 and 8000. The excipients in the present embodiments can include, for example, one or more of a polyethylene glycol, a 1,1,3,3-tetramethylbutyl)phenyl-polyethylene glycol, (2-hydroxylpropyl)-β-cyclodextrin, glycine, cellulose, citrate, lactose, mannitol, xylitol, sucrose, and polyvinylpyrrolidone. The reagent solution can be lyophilized and then specific amounts of the resulting powder can be added to each microwell of a microwell plate. In a preferred instance, a known volume of reagent solution is added to the microwells of the microwell plate and the microwell plate subjected to lyophilizing conditions.

In some preferred aspects, the composition in solution can be diluted and filtered to form an aqueous reagent solution having a composition of from about 0.05 to 0.5 mM indicator, preferable 0.1 mM to about 0.3 mM indicator, from about 5 mM to about 100 mM molybdate salt, preferably from about 25 mM to about 50 mM molybdate salt, from about 100 to about 500 mM of buffer, preferably from about 300 mM to about 400 mM buffer, from about 5 mM to 50 mM nitrate salt, preferably from about 10 mM to 25 mM, from 1 μg/L to 1000 mg/L sulfate salt, preferably from about 1 μg/L to 10 μg/L, and from about 1% to about 10% excipients. Acid (for example, hydrochloric acid) can be added to the solution to reduce the pH to a value of from about 1 to 2.5. For example, a 96-microwell plate can be filled with 50 to 275 microliters of aqueous reagent composition. Lyophilizing conditions include −60° C. to −40° at <200 mtorr.

In some other preferred aspects, the aqueous reagent solution comprises, consists essentially of, or consists of 0.2 mM malachite green, 40 mM ammonium molybdate, 375 mM of valine, 7 μg/L sodium sulfate, 15 mM potassium nitrate, and 7.5 wt/wt of (2-hydroxylpropyl)-β-cyclodextrin. Acid (for example, hydrochloric acid) can be added to the solution to reduce the pH to a value of preferably 1.75. Preferably, a 96-microwell plate can be filled with 150 microliters of aqueous reagent composition. Lyophilizing conditions are preferably −40° C. at 100 mtorr.

### B. Phosphate Assay Kit

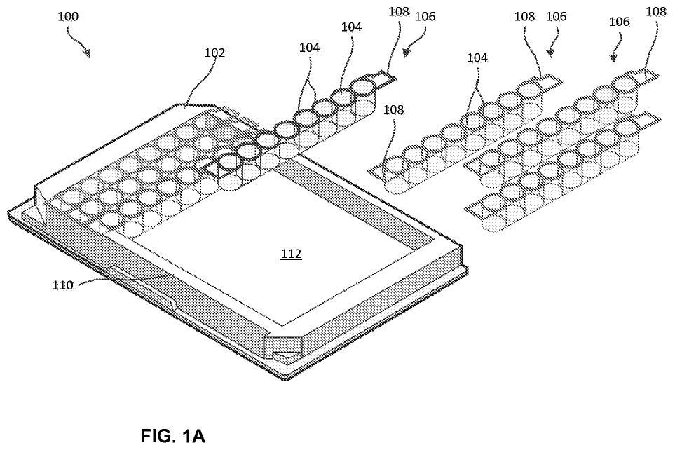

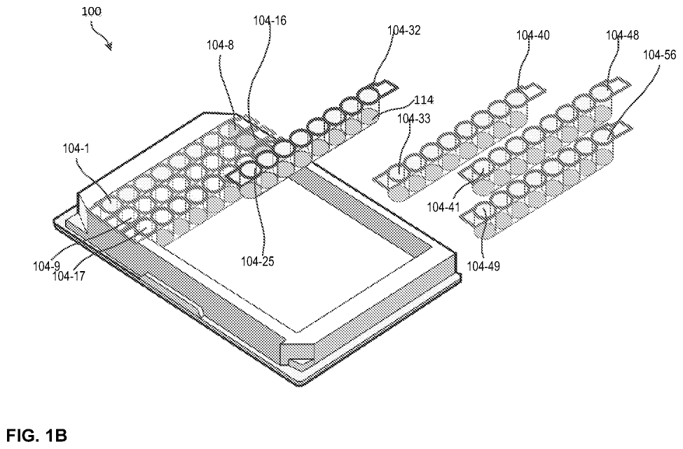

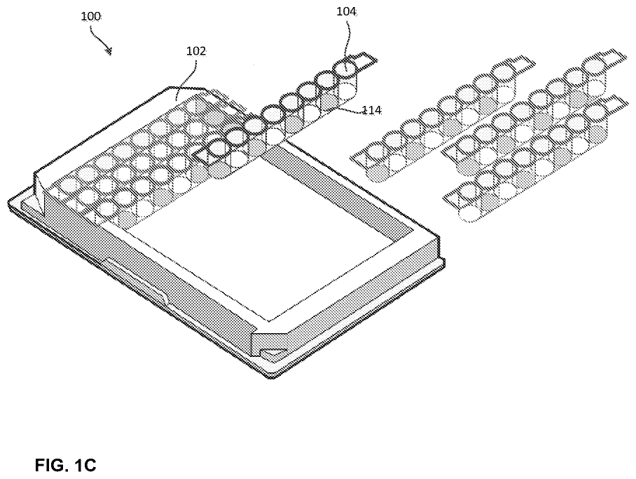

FIGS. 1A-1C depict schematics of embodiments of phosphate assay system 100. The phosphate assay system or kit includes microwell plate 102 having a plurality of microwells 104. The plurality of microwells 104 can be assembled in the removable holders 106. Holders 106 may include members 108 that position on top of the side wall 110. Holders 106 may rest on, or be suspended above, bottom wall 112 of the microwell plate 102. As shown, holder 106 includes eight (8) microwells 104, however, the number of microwells can be adjusted to the size of the microwell plate 102. For example, the number of the microwells 104 can be 2, 3, 4, 5, 6, 7, 8, 9, 10, 11, 12, 13, 14, 15, 16, 17, 18, 19, 20, 24, 48, 96, 384, 1536, or more, etc. As shown in FIG. 1A, the microwell plate 102 does not include any composition. FIG. 1B depicts all of the microwells having composition 114 and FIG. 1C depicts some of the microwells having composition 114. The microwells 104 can hold a volume of 20, 50, 300, 500 microliters, preferably 300 microliters or 400 microliters. The microwell plate 102, microwells 104, holders 106, can be made of any chemical resistant material. Non-limiting examples of materials include polymers, copolymers of polymers, polystyrene, polypropylene, cyclo-olefins and the like. The holders 106 may be polymeric or plastic tape with the microwells 104 embossed on the tape. Microwell plates are commercially available from Thermo Fisher Scientific (Waltham, Mass., USA).

As shown in FIG. 1B, the microwells 104 can be filled with the same amount of lyophilized composition. In other embodiments, the microwells 104 in each holder 106 can have the same amount of composition, but a total amount of composition in the holders 106 can be different. For example, microwells 104-1 to 104-8 can have a different amount of composition than microwells 104-9 to 104-16. It should be understood, that configuration of the amount composition in the microwells can be any chosen configuration that correlates to a calibration curve. In some instances, the microwells 104 are filled with a known amount of an aqueous solution of composition and then microwell plate is positioned in a lyophilizing unit and lyophilized under conditions sufficient to remove the water from the solution. The microwells 104, microwell holders 106, and/or the microwell plate can be sealed with a known sealing agent (for example, plastic film or foil) to allow the microwell plate 102 or the microwell holders 106 to stored or transported. In some embodiments, the phosphate assay system includes a spectrophotometer that measures the absorbance of the chosen colorimetric dye and/or a calibration curve. The calibration curve can depict the amount of phosphate versus absorbance value. In some instances, a calibration curve is provided for each holder 106.

### C. Method of Determining Phosphate

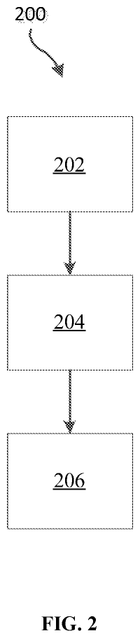

The phosphate assay system and kit described throughout the specification can be used to determine the phosphate concentration in an analyte composition. The analyte composition can be a sample from a water body such as a subsurface water well in a hydrocarbon formation, a wastewater storage unit, a wastewater reservoir, a lake, a river, a canal or the like. Referring to FIG. 2, a flow chart for determining phosphate concentration is depicted. In method 200, the microwell plate 102 containing the lyophilized composition 114 is obtained in step 202. In step 204, a known amount of analyte composition (for example 300 microliters) is added to the lyophilized composition 114 reagents in the microwells 104 using a delivery apparatus (for example, multichannel pipette). In step 206, after solids in the plate have fully dissolved, the microwell plate 102 is placed in a spectrophotometer (for example, a plate reader) and the absorbance at the known wavelength (for example 450 nm) for each microwell is measured. The phosphate concentration is determined by referring to a calibration curve and selecting the phosphate that correlates to the absorbance value.

The system 100 and processes of the present invention can be automated to acquire data. The acquired data can be transmitted to one or more computer systems. The computer systems can include components such as CPUs or applications with an associated machine readable medium or article which may store an instruction or a set of instructions that, if executed by a machine, may cause the machine to perform a method and/or operations in accordance with the methods of the present invention. For example, the microwell plate 102 can be put in a plate reader and the spectrophotometer can automatically measure the absorbance of each sample. The measured absorbance can be stored in a computer system in the spectrophotometer and/or transmitted to another computer system. Either computer may be capable of processing the absorbance and displaying or printing a phosphate value for a series of analytes. Such a machine may include, for example, any suitable processing platform, computing platform, computing device, processing device, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, and the like. The instructions may be implemented using any suitable high-level, low-level, object-oriented, visual, compiled and/or interpreted programming language, such as C, C++, Java, BASIC, Perl, Matlab, Pascal, Visual BASIC, assembly language, machine code, and so forth. The computer system may further include a display device such as monitor, an alphanumeric input device such as keyboard, and optionally a directional input device such as mouse. In some instances, a mobile computer such as a smart phone or tablet device can be used.

### EXAMPLES

The present invention will be described in greater detail by way of specific examples. The following examples are offered for illustrative purposes only and are not intended to limit the invention in any manner. Those of skill in the art will readily recognize a variety of noncritical parameters which can be changed or modified to yield essentially the same results.

### Example 1

### Phosphate Assay Kit

Compositions.

TABLE 1

| | for 1 L | | for 2 L | | for 5 L | | for 10 L | |
|---|---|---|---|---|---|---|---|---|
| Ammonium Molybdate | 7.72 | g | 15.44 | g | 38.60 | g | 77.20 | g |
| Malachite Green | 60 | mg | 120 | mg | 300 | mg | 600 | mg |
| Potassium Nitrate | 1.63 | g | 3.26 | g | 8.15 | g | 16.3 | g |
| Sodium Sulfate (1 mg/L solution) | 5 | mL | 10 | mL | 25 | mL | 50 | mL |
| (2-hydroxylpropyl)-β-cyclodextrin | 75 | g | 150 | g | 375 | g | 750 | g |
| Valine | 44.34 | g | 88.68 | g | 221.70 | g | 443.40 | g |

The reagents listed in Table 1 were combined in distilled water in and diluted to approximately 75% of the desired batch size. The pH was slowly adjusted to a pH of 1.75 using concentrated hydrochloric. Distilled water was added to increase the volume of the solution to approximately 95% of the batch size, the pH was monitored, and then the solution was transferred to an appropriately sized volumetric flask. The solution container was rinsed with small washes of distilled water and these rinses were transfer to the volumetric flask. The solution was then diluted to the desired volume (mark on the volumetric flask), and then filtered into an appropriate sized, clean media bottle using a bottle top or vacuum capsule filter, 0.20 microns. The valine can be increased in the Table 1 formulations. By way of example, the valine can be increased to 117.15 g (for 1 L), 234.30 g (for 2 L), 585.76 g (for 5 L), and 1,171.51 g (for 10 L).

The solution (150 μL) was added to microwells of a microwell plate. The aqueous composition was lyophilized to remove the water and a lyophilized sample in the microwell plate was obtained. Plates were first frozen at −40° C. followed by primary drying at −40° C. and 100 mtorr vacuum with steadily increasing temperature up to 20° C. until dry.

In an alternative embodiment, a phosphate assay composition can be made in view of the parameters set forth below in Table 2.

TABLE 2

| | |
|---|---|
| for 500 mL | for 1 L |

| | | | | |
|---|---|---|---|---|
| Ammonium Molybdate | 3.86 | g | 7.72 | g |
| Malachite Green (60 mg/10 mL) | 5 | mL | 10 | mL |
| Potassium Nitrate | 0.8065 | g | 1.613 | g |
| Sodium Sulfate (1 mg/L solution) | 2.5 | mL | 5 | mL |
| (2-hydroxylpropyl)-β-cyclodextrin | 37.5 | g | 75 | g |
| Valine | 58.5755 | g | 117.151 | g |

For the Table 2 compositions, valine and (2-hydroxylpropyl)-β-cyclodextrin were added to deionized (DI) water less than the total volume. The pH was adjusted to 1.75 using concentrated HCl. Ammonium molybdate, sulfate, and potassium nitrate were then added. If the solution turns cloudy, then the solution may be filtered with a filter paper. Malachite green was then added using a volumetric flask to dilute the solution to the final volume.

Calibration Curve. A calibration curve was prepared for the Table 1 compositions by diluting a phosphate standard (1000 mg/L NaH₂PO4, Sigma-Aldrich®) to the concentrations in Table 3, then filling a freeze-dried plate of a 150 microliter fill of the composition with 300 microliters of sample. Absorbance at 520 nm was found to be near an isosbestic point and is used as a blank. The absorbances are plotted as a function of the absorbances determined from the following equation:

(450 nm-520 nm)/(560 nm+595 nm+620 nm-3*520 nm)

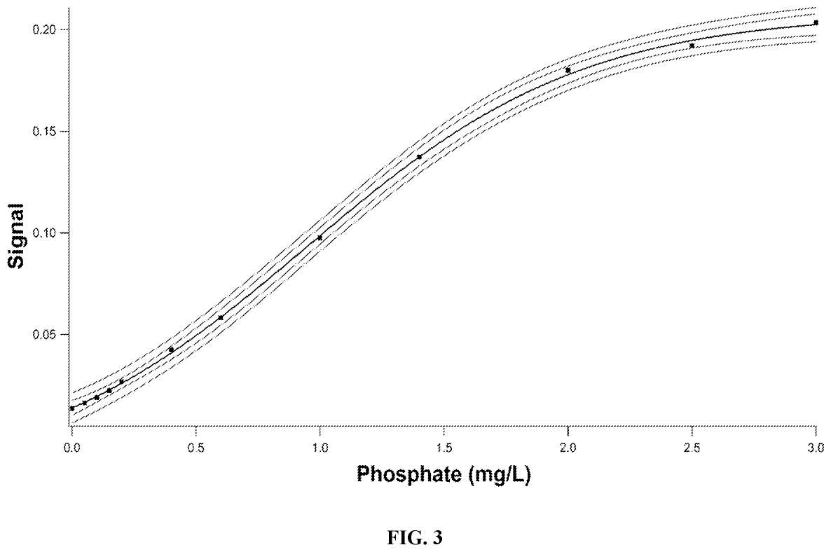

The data was then fit with a sigmoid equation as shown in FIG. 3.

TABLE 3

| PO₄ | Absorbance | % RSD |
|---|---|---|
| (mg/L) | Ratio | (OD) |
| 0 | 0.014 | 4.45 |
| 0.05 | 0.016 | 1.07 |
| 0.10 | 0.019 | 0.72 |
| 0.15 | 0.023 | 2.04 |
| 0.20 | 0.027 | 2.44 |
| 0.40 | 0.042 | 3.14 |
| 0.60 | 0.057 | 2.61 |
| 1.00 | 0.096 | 5.53 |
| 1.40 | 0.139 | 2.46 |
| 2.00 | 0.181 | 2.83 |
| 2.50 | 0.195 | 0.68 |
| 3.00 | 0.206 | 1.69 |

The lyophilized sample, the microwell plate and, optionally, a calibration curve or table constituted the phosphate assay kit.

### Example 2

### Determination of Phosphate in a Water Body

Phosphate Assay. Analyte compositions (300 microliters) containing an unknown amount of phosphate were added to 8 microwells of the 96-microwell plate prepared in Example 1, Table 1. After dissolution of the lyophilized sample, the microwell plate was positioned in a plate reader and the absorbance values of the plate was determined.

Table 4 shows how the current phosphate assay performs on samples from oil field water wells of varying composition. Samples were acidified and tested for phosphate concentration. Samples were then spiked with 1.0 mg/L phosphate and tested again. Of the 1.0 mg/L phosphate spike added to the samples, the average spike recovery was 1.05 mg/L. Standard deviation of the results is 0.09 mg/L.

TABLE 4

| | Sample | Sample + | Spike |
|---|---|---|---|
| | Result | 1.0 mg/L | Recovery |
| Sample ID | (mg/L) | PO4 | (mg/L) |
| #1 | 0.30 | 1.45 | 1.15 |
| #5 | 1.02 | 1.86 | 0.84 |
| #38 | 1.02 | 2.06 | 1.03 |
| #40 | 0.42 | 1.46 | 1.04 |
| #41 | 0.36 | 1.45 | 1.09 |
| #42 | 0.40 | 1.52 | 1.12 |
| #43 | 0.44 | 1.53 | 1.09 |
| #45 | 0.44 | 1.57 | 1.13 |
| #53 | 0.66 | 1.75 | 1.09 |
| #55 | 0.61 | 1.73 | 1.12 |
| #57 | 0.40 | 1.40 | 1.00 |
| #58 | 0.51 | 1.65 | 1.14 |
| #59 | 0.34 | 1.41 | 1.08 |
| #60 | 0.54 | 1.42 | 0.89 |
| #61 | 0.38 | 1.39 | 1.01 |

1. A composition for determining the phosphate concentration of a solution, the composition comprising an indicator, a molybdate salt, a buffer, a reaction accelerant, a sulfate salt, and one or more excipients, wherein the composition has an absorbance at a detectable wavelength in response to the phosphate concentration in the solution.

2. The composition of claim 1, wherein the indicator comprises one or more of methyl violet 2B, methyl violet 6B, methyl violet 10B, pararosaniline, fuchsine, new fuchsine, fuchsine acid, phenolphthalein, phenol red, chlorophenol red, cresol red, bromocresol purple, bromocresol green, bromophenol blue, pyrogallol red, malachite green, brilliant green, brilliant blue FCF, Victoria blue B, Victoria blue FBR, Victoria blue BO, Victoria blue FGA, Victoria blue 4 R, Victoria blue R, eosin B, eosin Y, rhodamine B, rhodamine 123, or fluorescein.

3. The composition of claim 2, wherein the indicator is malachite green.

4. The composition of any one of claims 1 to 3, wherein the molybdate salt comprises one or more of ammonium molybdate, ammonium molybdate tetrahydrate, sodium molybdate, sodium molybdate dihydrate, ceric ammonium molybdate, cerium molybdate, potassium molybdate, magnesium molybdate, lithium molybdate, calcium molybdate, zinc molybdate, bismuth molybdate, lead molybdate, molybdic acid, nickel molybdate, silver molybdate, strontium molybdate, barium molybdate, or cadmium molybdate.

5. The composition of claims 1 to 4, wherein the molybdate salt is ammonium molybdate.

6. The composition of any one of claims 1 to 5, wherein the buffer comprises one or more of camphorsulfonate, camphorsulfonic acid, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, selenocysteine, pyrrolysine, or valine.

7. The composition of claim 6, wherein the buffer is valine.

8. The composition of any one of claims 1 to 7, wherein the reaction accelerant comprises a nitrate salt such as one or more of sodium nitrate, lithium nitrate, nitric acid, potassium nitrate, magnesium nitrate, calcium nitrate, strontium nitrate, barium nitrate, or ammonium nitrate.

9. The composition of claim 8, wherein the reaction accelerant is potassium nitrate.

10. The composition of claim 9, wherein the sulfate salt comprises one or more of sodium sulfate, potassium sulfate, lithium sulfate, sulfuric acid, magnesium sulfate, or calcium sulfate.

11. The composition of claim 10, wherein the sulfate salt is sodium sulfate.

12. The composition of any one of claims 1-11, further comprising a sample pretreatment neutralizer comprising one or more of ascorbic acid, sodium ascorbate, sodium nitrite, potassium nitrite, sodium sulfite, potassium sulfite, ferrous iron salt, glutathione, dithiothreitol, a reducing sugar, and a free radical scavenger.

13. The composition of claim 12, wherein the sample pretreatment neutralizer is ascorbic acid or sodium ascorbate.

14. The composition of claim 13, further comprising a sample pretreatment neutralizer catalyst comprising one or more of a ferrous iron salt, a ferric iron salt, and a transition metal complex such as Fe(II)-EDTA complex, Fe(III)-EDTA complex, Fe(II)-CDTA complex, or Fe(III)-CDTA complex.

15. The composition of claim 14, wherein the sample pretreatment neutralizer catalyst is Fe(III)-EDTA complex.

16. The composition of claim 15, wherein the sulfate salt is sodium sulfate.

17. The composition of any one of claims 1 to 16, wherein the excipients comprise one or more of a polyethylene glycol, a 1,1,3,3-tetramethylbutyl) phenyl-polyethylene glycol, (2-hydroxylpropyl)-β-cyclodextrin, glycine, cellulose, citrate, lactose, mannitol, xylitol, sucrose, or polyvinylpyrrolidone.

18. The composition of claim 17, wherein the excipient is (2-hydroxylpropyl)-β-cyclodextrin.

19. The composition of any one of claims 1 to 18, wherein the composition is a powder.

20. The composition of claim 19, wherein the powder is made by providing an aqueous solution of the composition to one or more containers and subjecting at least one of the containers to lyophilizing conditions sufficient to remove the water from the aqueous solution to form the powder.

21. The composition of claim 20, wherein the one or more containers are microwells of a microwell plate.

22. The composition of claim 20, wherein the container is a bag or a vial.

23. The composition of claim 1, wherein the composition is lyophilized and consists essentially of or consists of ammonium molybdate as the molybdate salt, malachite green as the indicator, potassium nitrate as the reaction accelerant, sodium sulfate as the sulfate salt, (2-hydroxylpropyl)-β-cyclodextrin as the excipient, and valine as the buffer.

24. A phosphate assay kit comprising:
a) a microwell plate; and
b) the composition of any one of claims 1 to 23;
wherein a plurality of microwells of the microwell plate contain the lyophilized composition such that when an analyte composition is added to the lyophilized composition in each microwell of the plurality of microwells a solution forms having an absorbance at a detectable wavelength in response to the phosphate concentration of an aqueous solution.

25. The phosphate assay kit of claim 24, wherein the microwell plate comprises 6, 24, 96, 384, or 1536 microwells.

26. The phosphate assay kit of claim 24, wherein the microwell plate comprises 6 microwells and each microwell contains the same amount of composition.

27. The phosphate assay kit of claim 24, wherein the microwell plate comprises 24 microwells or 96 microwells.

28. The phosphate assay kit of claim 24, wherein the microwell plate comprises 6 microwells, wherein at least 2 microwells have the same composition and wherein at least 2 microwells have different amounts of the same composition or different amounts of the same components of the composition.

29. The phosphate assay kit of claim 24, wherein the microwell plate comprises 24 microwells or 96 microwells, wherein at least 2 microwells have the same composition and wherein at least 2 microwells have different amounts of the same composition or different amounts of the same components of the composition.

30. The phosphate assay kit of any one of claims 24 to 29, wherein the indicator comprises one or more of methyl violet 2B, methyl violet 6B, methyl violet 10B, pararosaniline, fuchsine, new fuchsine, fuchsine acid, phenolphthalein, phenol red, chlorophenol red, cresol red, bromocresol purple, bromocresol green, bromophenol blue, pyrogallol red, malachite green, brilliant green, brilliant blue FCF, Victoria blue B, Victoria blue FBR, Victoria blue BO, Victoria blue FGA, Victoria blue 4 R, Victoria blue R, eosin B, eosin Y, rhodamine B, rhodamine 123, or fluorescein.

31. The phosphate assay kit of claim 24, wherein the indicator is malachite green.

32. The phosphate assay kit of any one of claims 24 to 31, wherein the molybdate salt comprises one or more of ammonium molybdate, ammonium molybdate tetrahydrate, sodium molybdate, sodium molybdate dihydrate, ceric ammonium molybdate, cerium molybdate, potassium molybdate, magnesium molybdate, lithium molybdate, calcium molybdate, zinc molybdate, bismuth molybdate, lead molybdate, molybdic acid, nickel molybdate, silver molybdate, strontium molybdate, barium molybdate, or cadmium molybdate.

33. The phosphate assay kit of claim 32, wherein the molybdate salt is ammonium molybdate.

34. The phosphate ion assay kit of any one of claims 24 to 33, wherein the buffer comprises one or more of camphorsulfonate, camphorsulfonic acid, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, selenocysteine, pyrrolysine, or valine.

35. The phosphate ion assay kit of claim 34, wherein the buffer valine.

36. The phosphate ion assay kit of any one of claims 24 to 35, wherein the reaction accelerant comprises a nitrate salt such as one or more of sodium nitrate, lithium nitrate, nitric acid, potassium nitrate, magnesium nitrate, calcium nitrate, strontium nitrate, barium nitrate, or ammonium nitrate.

37. The phosphate ion assay kit of claim 36, wherein the reaction accelerant is potassium nitrate.

38. The phosphate ion assay kit of any one of claims 24 to 37, wherein the sulfate salt comprises one or more of sodium sulfate, potassium sulfate, lithium sulfate, sulfuric acid, magnesium sulfate, or calcium sulfate.

39. The phosphate ion assay kit of claim 38, wherein the sulfate salt is sodium sulfate.

40. The phosphate ion assay kit of any one of claims 24 to 39, wherein the excipients comprise one or more of a polyethylene glycol, a 1,1,3,3-tetramethylbutyl)phenyl-polyethylene glycol, (2-hydroxylpropyl)-β-cyclodextrin, glycine, cellulose, citrate, lactose, mannitol, xylitol, sucrose, trehalose, or polyvinylpyrrolidone.

41. The phosphate ion assay kit of claim 40, wherein the excipient is (2-hydroxylpropyl)-β-cyclodextrin.

42. The phosphate ion assay kit of any one of claims 24 to 41, wherein the lyophilized composition consists essentially of or consists of ammonium molybdate, malachite green, potassium nitrate, sodium sulfate, (2-hydroxylpropyl)-β-cyclodextrin, and valine.

43. The phosphate ion assay kit of any one of claims 24 to 42, wherein the detectable wavelength is between 400 and 700 nm.

44. The phosphate ion assay kit of any one of claims 24 to 43, wherein the plurality of microwells are sealed to prevent the composition from exiting the plurality of microwells.

45. The phosphate ion assay kit of claim 44, wherein the plurality of microwells are sealed with a plastic film or a foil.

46. The phosphate assay kit of any one of claims 24 to 45, further comprising a spectrophotometer capable of measuring ultraviolet and/or visible wavelengths.

47. A method of determining a phosphate concentration of an analyte composition, the method comprising:
a) obtaining any one of the phosphate assay kits of claims 24 to 46;
b) obtaining an analyte composition;
c) adding substantially the same volume of the analyte composition to each of the plurality of microwells of the microwell plate to form solutions from the analyte composition and the lyophilized compositions in each of the plurality of microwells; and
d) measuring the absorbance value for each solution in each of the plurality of microwells at a wavelength and determining the phosphate concentration of the analyte composition based on the measured absorbance values.

48. The method of claim 47, wherein the composition consists essentially of or consists of ammonium molybdate, malachite green, potassium nitrate, sodium sulfate, (2-hydroxylpropyl)-β-cyclodextrin, and valine.

49. The method of any one of claims 47 to 49, wherein the analyte is an aqueous composition obtained from a subsurface well.

50. The method of any one of claims 47 to 49, wherein the analyte composition comprises a plurality of solutions having the same analyte, and each analyte composition is obtained from a different well of a plurality of subsurface wells.

51. The method of any one of claims 47 to 49, wherein the aqueous composition comprises an aqueous solution or suspension such as blood, plasma, lymph, urine, saliva, milk, juice, beer, wine, spirits, liqueur, soft drink, sports drink, soup, stew, broth, bottled water, tap water, tea, coffee, electrolyte solution, saline solution, hydraulic fracturing fluid, chemical formulation, coolant, cleaning solution, aqueous extract, fertilizer solution, culture media, rainwater, agricultural runoff, ballast water, bilge water, mining runoff, food processing waste water, blowdown water, brackish water, municipal drinking water, or the like, or any mixture of these solutions or suspensions.

52. The method of any one of claims 47 to 51, wherein the aqueous composition is an aqueous sample obtained from any source.

53. The method of any one of claims 47 to 51, wherein the aqueous composition is an aqueous sample obtained from a water treatment facility, mine, drilling process, hydraulic fracturing process, port, port authority, dock, subsurface water well in a hydrocarbon formation, hydrocarbon well, aquifer, wastewater storage unit, a boiler, cooling unit, wastewater reservoir, tank, water well, lake, river, ocean, canal, pond, pool, or the like, or any combination of these sources.

54. The method of any one of claims 47 to 51, wherein the aqueous composition is an aqueous sample obtained from a subsurface water source such as an aquifer, water well, formation, hydrocarbon well, or the like.

55. The method of any one of claims 47 to 51, wherein the aqueous composition is an aqueous sample obtained from a surface water source such as a lake, river, ocean, canal, pond, pool, or the like.

56. The method of any one of claims 47 to 51, wherein the aqueous composition is an aqueous sample obtained from an industrial site such as a boiler, cooling unit, wastewater reservoir, tank, or the like.

57. The method of any one of claims 47 to 51, wherein the aqueous composition is an aqueous sample obtained from a water treatment facility.

58. The method of any one of claims 47 to 51, wherein the aqueous composition is an aqueous sample obtained from a port or port authority.

59. The method of claims 47 to 51, wherein the well is a hydrocarbon well or a water well in a subsurface hydrocarbon formation.

60. The method of any one of claims 47 to 51, wherein the analyte composition is obtained from a drilling process or hydraulic fracturing process.

61. The method of any one of claims 47 to 51, wherein the analyte composition is obtained from a wastewater tank or reservoir.

62. A method of making any one of the phosphate assay kits of claims 24 to 46, the method comprising:
a) obtaining a microwell plate; and
b) obtaining a lyophilized composition comprising an indicator, a molybdate salt, a buffer, a reaction accelerant, a sulfate salt, and one or more excipients wherein a plurality of microwells of the microwell plate contain the lyophilized composition such that when an analyte composition is added to the lyophilized composition in each microwell of the plurality of microwells a solution forms having an absorbance at a detectable wavelength in response to phosphate comprised in the analyte composition.

63. The method of claim 62, wherein obtaining a lyophilized composition comprises providing an aqueous solution of the composition to one or more microwells of the microwell plate and subjecting the microwell plate to lyophilizing conditions sufficient to remove the water from the aqueous solution and form a powder.

64. The method of any one of claims 62 to 63, wherein the plurality of microwells are sealed to prevent the lyophilized composition from exiting the plurality of microwells.

65. The method of claim 64, wherein the plurality of microwells are sealed with a plastic film or a foil.
